(19) United States
(12) Patent Application Publication
(10) Pub. No.: US 2017/0165475 A1
(43) Pub. Date: Jun. 15, 2017

(54) Electrode Implantation System

(71) Applicant: BIOTRONIK SE & Co. KG, Berlin (DE)

(72) Inventors: Dajana Kaiser, Berlin (DE); Detmar Jadwizak, Erkner (DE); Carsten Fruendt, Berlin (DE); Gordon Hillebrand, Berlin (DE)

(21) Appl. No.: 15/370,263

(22) Filed: Dec. 6, 2016

(30) Foreign Application Priority Data
Dec. 14, 2015 (DE) 10 2015 121 726.0

(51) Int. Cl. A61N 1/05; A61B 17/34

(52) U.S. Cl. CPC A61N 1/056; A61B 17/3468

(57) ABSTRACT

An electrode implantation system including:
A) At least one elongated implantation tool that comprises a proximal section that is, at least in sections, in the form of a mandrel, and a distal section that is in the form of a flexible guide wire, the proximal and the distal sections being delimited from one another by a static or adjustable stop element comprising a stop surface facing the distal section; and
B) An implantable electrode with an electrode sleeve that has a stop area that is complementary to the stop surface and that faces the proximal section.

The electrode sleeve and the elongated implantation tool are arranged so that they are, at least in sections, coaxial with one another, and can be moved relative to one another along and around a common longitudinal axis. Other aspects relate to an elongated implantation tool and an implantable electrode.

### CROSS-REFERENCE TO RELATED APPLICATIONS

This patent application claims the benefit of and priority to co pending German Patent Application No. DE 10 2015 121 726.0, filed on Dec. 14, 2015 in the German Patent Office, which is hereby incorporated by reference in its entirety.

### TECHNICAL FIELD

This invention relates to an electrode implantation system, an elongated implantation tool, and an implantable electrode.

### BACKGROUND

Cardiovascular diseases are among the most serious diseases of modern society. Even today, many diseases still have a fatal course. Especially older persons are affected by cardiovascular diseases. Therefore, in view of rising life expectancy and the growing number of chronic heart diseases, it can be expected that there will be a further increase in these diseases. The main causes, which are cited again and again, are widespread influencing factors of a modern and globally integrated society such as stress, smoking, excessively fatty food, and the accompanying overweight or high blood pressure. But genetic predisposition or viral infections can also cause heart diseases. Since cardiovascular system disorders already manifest themselves in the younger years, and especially since younger people are growing up from the very beginning in an environment favoring these influencing factors, a further intensification of the trend toward cardiovascular diseases should be expected. Therefore, an important starting point is a consistent improvement in medical care.

This leads first to rising costs and second also to higher requirements on the safety of medical technology products, for example, cardiac pacemakers, since the future trend is toward an increasing number of these systems being in circulation, and the safety against malfunctions must be correspondingly high.

Non-homogeneous contraction of the individual areas of the left ventricle can cause heart failure, also called cardiac insufficiency. The main reason for non-homogeneous, or asynchronous contraction, is a disturbance in the electrical conduction system of the heart. So-called cardiac resynchronization therapy, CRT stimulation for short, makes the contraction of the left ventricle homogenous again, or simply put it resynchronizes it, allowing the heart to recover its pumping force again. As a rule, the process involves the use of two electrodes. One electrode is implanted in the right ventricle. The second electrode is anchored to a branch of the coronary sinus of the left ventricle, and thus lies on the outside of the left ventricle. Simultaneously delivering pulses through both electrodes once again causes synchronous electrical stimulation of the left part of the heart and allows homogeneous contraction.

One possible way of bringing the electrode to its destination is the so-called “over-the-wire technique”, or the OTW technique for short. This involves guiding the electrode over a guide wire all the way to the desired target vein. The guide wire is pushed into and through the electrode. The guide wire can be introduced into the electrode from the distal direction or from the proximal direction. The guide wire is then used to search for a path through the individual vessels to the target vein, and after that the electrode is guided over the guide wire. After reaching the target vein, the electrode must be securely fixed in the vessel. In the case of CRT electrodes, this is usually made possible by passive fixation elements of the electrode. These can be in the form of soft outer contours such as, for example, screw threads or knobs. However, they can also be in the form of shaped distal areas, such as, for example, J-curves, S-curves, or anchor-like structures. The soft outer contours allow jamming and fixation of the electrode in the so-called wedge position. The shaped distal areas allow positioning of the electrode in larger vessels by jamming its curves or anchor-like structures against the vessel wall, and thus holding the electrode in position. To accomplish this, the guide wire is conventionally pulled out of the electrode and replaced by a stiffer mandrel. Using this mandrel it is possible to jam in the electrode. Such a mandrel can also be used to align the electrode at the beginning of the operation and introduce it into the body.

It turns out to be problematic that the stiffness of the mandrel strongly hinders the guidance of the electrode to the target vein. Therefore, as a rule, it is replaced by a guide wire after the electrode is introduced into the body. Another problem turns out to be that the small stiffness of the guide wire makes it scarcely possible to jam the electrode in the target vein over the guide wire. Thus, it is necessary to change back to the mandrel again. However, when this is done the electrode frequently slips. Slipping of the electrode can also occur when the mandrel is guided into the electrode to perform the jamming. As a consequence, an iterative alternating use of guide wire and mandrel can be necessary. These difficulties increase the duration of the operation, and thus also the costs and the risk for the patient.

The present invention is directed toward overcoming one or more of the above-mentioned problems.

### SUMMARY

One or more of the portrayed disadvantages of the prior art are remedied or at least reduced with the help of the invention. To accomplish this, a first aspect of the present invention relates to an electrode implantation system comprising:

A) At least one elongated implantation tool that comprises a proximal section that is, at least in sections, in the form of a mandrel, and a distal section that is in the form of a flexible guide wire, the proximal and the distal sections being delimited from one another by a static or adjustable stop element comprising a stop surface facing the distal section; and

B) An implantable electrode with an electrode sleeve that has a stop area that is complementary to the stop surface and that faces the proximal section.

The electrode sleeve and the elongated implantation tool are arranged so that they are, at least in sections, coaxial with one another, and can be moved relative to one another along and around a common longitudinal axis.

The electrode implantation system offers the advantage that implantation of the electrode is possible without this requiring switching back and forth between different tools. Thus, the electrode implantation system allows a simple, safe, and rapid implantation of the electrode. Thus, the elongated implantation tool can first be introduced into the electrode sleeve or guided through it. The distal section of the elongated implantation tool in the form of a guide wire can then be introduced into the patient's body, and be used for navigation through the vessels. The elongated implantation tool can be pushed through the electrode sleeve until the stop surface of the stop element comes to lie against the complementary stop area of the electrode sleeve. In this state, sections of the electrode are located on the proximal section in the form of a mandrel, and the electrode is aligned and can be introduced into the patient's body in a simple manner.

In other embodiments, the electrode can also be pushed beyond the stop element, completely onto the proximal section. Such embodiments of the electrode implantation systems will still be described below. Then, the electrode can be moved back in the direction of the distal section onto the elongated implantation tool, and navigated over it to the target vein. In the target vein, the elongated implantation tool with the stop surface can then be brought to lie against the complementary stop area of the electrode sleeve again, and a jamming force can be transferred onto the electrode sleeve. After the electrode has been securely fixed in the vessel, the elongated implantation tool can be withdrawn, and removed from the electrode and finally out of the patient's body.

In connection with this invention, the term ‘proximal’ is defined as pointing in the direction of the middle of the body of a user of the electrode implantation system. The term user designates an operator, that is, as a rule, a doctor or operating surgeon. Accordingly, the term “distal” is defined as pointing opposite the direction of the middle of the body of the user of the electrode implantation system. In other words, the term distal designates a direction toward the patient. These definitions apply analogously if the elongated implantation tool or the electrode or the electrode sleeve are mentioned by themselves.

The term of the “common longitudinal axis” relates to that area in which the elongated implantation tool is concentrically arranged in the electrode sleeve. The electrode sleeve has dimensionally stable properties. Thus, the elongated implantation tool, at least components of which have elastic properties or flexible properties, fits to the shape of a through hole in the electrode sleeve. In this area, a longitudinal axis of the electrode sleeve and a longitudinal axis of the elongated implantation tool are collinearly aligned with one another, and thus are identical. The person skilled in the art is aware without a second thought that the elongated implantation tool can assume elastic lines independent of the electrode sleeve, similar to a hose, a small thin tube, or a thin wire. Its longitudinal axis can then also represent a curved line. If the discussion below refers to the longitudinal axis of the elongated implantation tool by itself, this relates to a state in which the entire elongated implantation tool, or a relevant section of it, is linearly stretched out.

A first variant of a preferred embodiment of the inventive electrode implantation system provides that the stop element comprises a rotationally symmetric area around the common longitudinal axis, this rotationally symmetric area having a larger cross section transverse to the common longitudinal axis than the distal section does, and in each of the relative orientations of the elongated implantation tool and the electrode sleeve around the common longitudinal axis a projection of the complementary stop area along the common longitudinal axis overlaps with the stop surface.

In other words, the elongated implantation tool according to the preceding embodiment has a body of revolution formed around its longitudinal axis, this body of revolution increasing the cross section of the elongated implantation tool. Preferably, the proximal section and the distal section are also in the form of a body of revolution. The distal section can preferably have a length of 10 mm to 300 mm, in particular 20 mm to 200 mm. A cross-sectional diameter of the stop element is always larger than a cross-sectional diameter of the distal section. The cross-sectional diameter of the stop element can be greater than or equal to that of the proximal section. If the electrode sleeve with the complementary stop area is laterally moved along the common longitudinal axis in the direction of the stop element, then its complementary stop area comes to lie against the latter. In this embodiment, this does not depend on how the electrode sleeve and the elongated implantation tool are rotated relative to one another around the common longitudinal axis. That is, the stop element will limit the lateral mobility of the electrode on one side of the elongated implantation tool. The inventive electrode implantation system offers the advantage that it is built in an especially simple manner and thus it is economical to produce and simple to operate.

Another preferred embodiment of the previously described first variant of the electrode implantation system provides that the stop element is in the form of a spherical or ring-shaped thickening of the elongated implantation tool. This offers the advantage that it avoids abrupt changes in cross section and, for example, sharp edges on the stop element. Thus, it is especially simple to introduce the stop element into the electrode sleeve as far as the complementary stop area.

A second variant of the electrode implantation system provides that the stop element comprises a rotationally asymmetric area around the common longitudinal axis, this rotationally asymmetric area having a larger cross section transverse to the common longitudinal axis than the distal section does, and that there is a limited number of relative orientations of the elongated implantation tool and the electrode sleeve around the common longitudinal axis in which a projection of the complementary stop area along the common longitudinal axis does not overlap with the stop surface in any place.

The term ‘rotationally asymmetric area’ can be illustrated by first imagining a rotationally symmetric area from sections of which material is then removed along the longitudinal axis of the elongated implantation tool. The material is removed from the outside toward the inside in the radial direction relative to the longitudinal axis of the elongated implantation tool. This gives the rotationally symmetric area different cross-sectional diameters in the radial direction. The complementary stop area of the electrode sleeve is now shaped in such a way that the rotationally asymmetric area of the stop element can, for example, in a single relative rotational orientation of the elongated implantation tool and the electrode sleeve around the common longitudinal axis, be pushed laterally along the common longitudinal axis through the complementary stop area of the electrode sleeve. By contrast, in all other relative rotational orientations, the stop surface of the stop element comes to lie against the complementary stop area. This offers the advantage that it is possible to switch between two states by changing the relative rotational orientation of the elongated implantation tool and the electrode. In a first state, the electrode and the elongated implantation tool can be moved relative to one another along the common longitudinal axis over the entire length of the elongated implantation tool. In a second state, the travel from distal to proximal is limited by the stop element and the complementary stop area. This significantly increases the flexibility of operation of the electrode implantation system. For example, the elongated implantation tool can be introduced into the electrode sleeve from two selectable directions. In this embodiment, the functionality of the stop element and the complementary stop area can be described with a lock-and-key principle. The distal section can preferably have a length of 10 mm to 60 mm, in particular 20 mm to 50 mm. The size of the cross section of the proximal section can also increase from the proximal direction in the distal direction all the way to the stop element, and can do so continuously or in the form of a profile. This facilitates centering of the electrode sleeve on the elongated implantation tool in the direction of the stop element. This significantly simplifies the lock-and-key principle.

Another preferred embodiment of the second variant provides that the stop element is in the form of a thickening of the elongated implantation tool, the shape of this thickening's cross section transverse to the common longitudinal axis being polygonal or elliptical in the area of the stop surface. This offers the advantage that the cross sectional shapes can be produced with little effort and are simple to manage using the lock-and-key principle.

Another, third variant of the inventive electrode implantation system provides that the elongated implantation tool is made in two parts and comprises an inner part in the form of a flexible guide wire and an outer part in the form of a mandrel, and that the outer part is arranged so that an inner lateral surface of it can be moved on an outer lateral surface of the inner part, so that the distal section of the elongated implantation tool is formed by the inner part and the proximal section is formed by the inner part and the outer part, the outer part comprising the stop element.

Thus, in this embodiment the respective lengths of the proximal and distal sections can be flexibly adjusted. The outer part, which is in the form of a mandrel, is pushed onto the inner part, which is in the form of a guide wire. The respective lengths of the distal section, which is in the form of a guide wire, and the proximal section, which is in the form of a guide wire and a mandrel surrounding it, can be flexibly adjusted according to how far the outer part is pushed onto the inner part. The inner part can be, for example, in the form of a conventional guide wire with a diameter of 0.36 mm, for example. The outer part can be, for example, in the form of a flexible hollow cylindrical element with, for example, an inside diameter of 0.38 mm and an outside diameter of 0.48 mm. The inner and outer part can be made of the same material or of different materials.

In the previously mentioned variant, the stop surface of the stop element is preferably located on a distal end area of the outer part. This embodiment offers the advantage that the length of the distal section, and thus the available length of the guide wire, can be flexibly adjusted. This is especially advantageous for navigation of the electrode in strongly branched and bent vessels. Another advantage of this embodiment is that the inner part in the form of a guide wire can be pulled completely out of the outer part once the electrode has reached its destination and the electrode can be jammed in place with the outer part. This makes the electrode implantation system especially simple to operate and especially flexible. Since the individual elements of the elongated implantation tool can be produced separately from one another, there are additional advantages with respect to production expense and quality, for example, in the case of larger series production.

Another preferred embodiment of the third variant of the inventive electrode implantation system provides that the outer part has a tubular or helical geometry and the stop surface is provided on a distal end area of the tubular or helical geometry. Such geometries can advantageously be produced with little expense, and are suitable for simple and flexible operation. The greatest variety of different mechanical properties can be flexibly produced by corresponding shaping of the tubular or helical geometry. For example, the tubular geometry can be closed, that is, in the form of a hollow cylinder. However, hollow cylindrical geometries are also possible in which the lateral surface has, for example, oblong slots extending along the longitudinal axis of the tubular geometry. Also possible are oblong slots in the lateral surface, each of which extends on a circumference or part of a circumference around the lateral surface. With respect to the helical geometry, it is possible to vary the pitch, for example. For example, the pitch can be constant or have a pitch profile over the longitudinal extension of the helical geometry. In particular, this allows a stiffness profile of the outer part to be flexibly adjusted to the requirements of the operation and the patient.

Basically, for all embodiments of the inventive electrode implantation system, it can be said that it is advantageous for a stiffness profile of the mandrel to have higher stiffness in the proximal direction and lower stiffness in the distal direction. This way, the further the operating surgeon advances the electrode implantation system into the patient's body, the better the capability of targeted navigation with the distal section is preserved. Other stiffness properties of the mandrel and the guide wire and the elongated implantation tool as a whole are selected by the responsible person skilled in the art himself. In particular, he takes into consideration the requirements presented by the operation process and the patient. All materials that are known to be suitable for guide wires and mandrels are possible materials for the elongated implantation tool.

Another aspect of this invention relates to an elongated implantation tool comprising:

A proximal section, at least sections of which are in the form of a mandrel;

A distal section that is in the form of a flexible guide wire; and

A static or adjustable stop element that delimits the proximal and the distal sections from one another and that comprises a stop surface facing the distal section.

A preferred embodiment of the elongated implantation tool provides that the stop element comprises a rotationally symmetric area around a longitudinal axis of the elongated implantation tool, this rotationally symmetric area having a larger cross section transverse to the longitudinal axis than the distal section does.

Another preferred embodiment of the elongated implantation tool provides that the stop element comprises a rotationally asymmetric area around a longitudinal axis, this rotationally asymmetric area having a larger cross section transverse to the longitudinal axis than the distal section does. In particular, the stop element is in the form of a thickening of the elongated implantation tool, the shape of this thickening's cross section transverse to the longitudinal axis being polygonal or elliptical in the area of the stop surface.

Another preferred embodiment of the elongated implantation tool provides that it is made in two parts and comprises an inner part in the form of a flexible guide wire and an outer part in the form of a mandrel, and that the outer part is arranged so that an inner lateral surface of it can be moved on an outer lateral surface of the inner part, so that the distal section is formed by the inner part and the proximal section is formed by the inner part and the outer part, the outer part comprising the stop element. In particular, the outer part has a tubular or helical geometry, and the stop surface is provided on a distal end area of the tubular or helical geometry.

Another aspect of this invention relates to an implantable electrode comprising an electrode sleeve that has a stop area extending transverse to a longitudinal axis of the electrode sleeve. A preferred embodiment of the implantable electrode provides that the stop area has a polygonal or elliptical cutout.

Other preferred embodiments of the inventive electrode implantation system, the inventive elongated implantation tool, and the inventive implantable electrode follow from the following description.

Also disclosed is a technical teaching on the execution of a process for implantation of an electrode.

Further embodiments, features, aspects, objects, advantages, and possible applications of the present invention could be learned from the following description, in combination with the Figures, and the appended claims.

### DESCRIPTION OF THE DRAWINGS

This invention is explained in detail below on the basis of some sample embodiments. The figures are as follows:

FIGS. 1A-1B show a representation of the principles of a first variant of the inventive electrode implantation system;

FIGS. 2A-2C show a representation of the principles of a second variant of the inventive electrode implantation system; and

FIGS. 3A-3G show a representation of the principles of a third variant of the inventive electrode implantation system.

### DETAILED DESCRIPTION

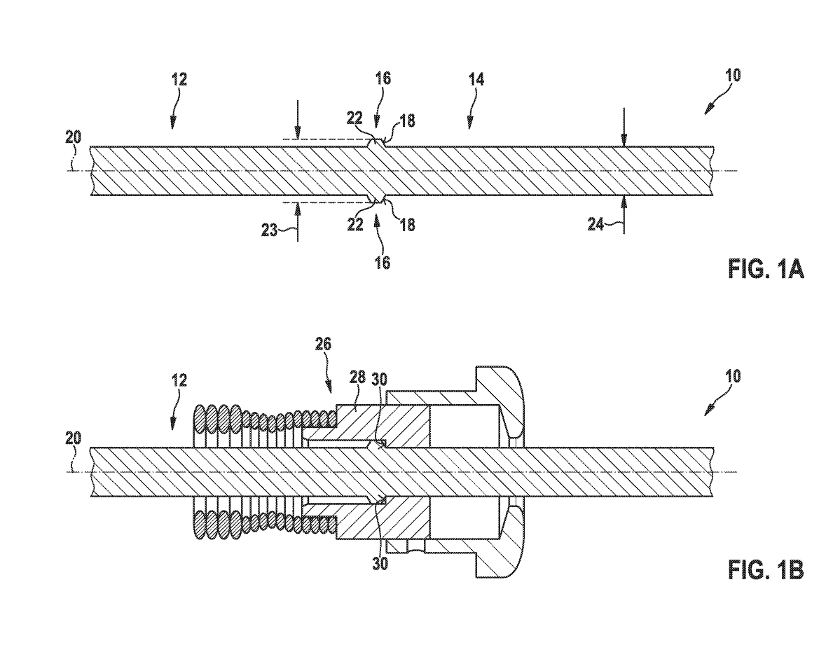

FIGS. 1A-1B show two sectional views through areas of a first variant of the electrode implantation system that are essential to the present invention. FIG. 1A shows an elongated implantation tool 10 that comprises a proximal section 12 and a distal section 14 that are, in this sample embodiment, delimited from one another by a static stop element 16. The proximal section 12 is in the form of a mandrel and has higher stiffness properties than the distal section 14, which is in the form of a flexible guide wire.

The stop element 16 has a stop surface 18 that faces the distal section 14. In this sample embodiment, the stop element 16 is in the form of a ring-shaped thickening of the elongated implantation tool 10. That is, the stop element 16 comprises a rotationally symmetric area 22 formed around its longitudinal axis 20, this rotationally symmetric area 22 having a cross section 23 transverse to a longitudinal axis 20 that is larger than a cross section 24 of the distal section 14.

FIG. 1B shows the elongated implantation tool 10 with sections of the implantable electrode 26. The implantable electrode 26 comprises an electrode sleeve 28. The electrode sleeve 28 and the elongated implantation tool 10 are arranged coaxially with one another. This means that the electrode sleeve 28 and the elongated implantation tool 10 have a common longitudinal axis 20, at least over the length of the electrode sleeve 28. The electrode sleeve 28 and the implantation tool 10 can be moved relative to one another transversely along and rotationally around this common longitudinal axis 20.

The electrode sleeve 28 has a stop area 30 that is complementary to the stop surface 18. The stop area 30 faces the proximal section 12. In every possible relative orientation of the elongated implantation tool 10 and the electrode sleeve 28 around the common longitudinal axis 20, a projection of the complementary stop area 30 in the direction of the stop surface 18 and along the common longitudinal axis 20 overlaps with the stop surface 18. In other words, travel of the electrode 26 on the elongated implantation tool 10 in the direction of the proximal section 12 is limited by the fact that there is mating between the effective area of the stop surface 18 and that of the complementary stop area 30.

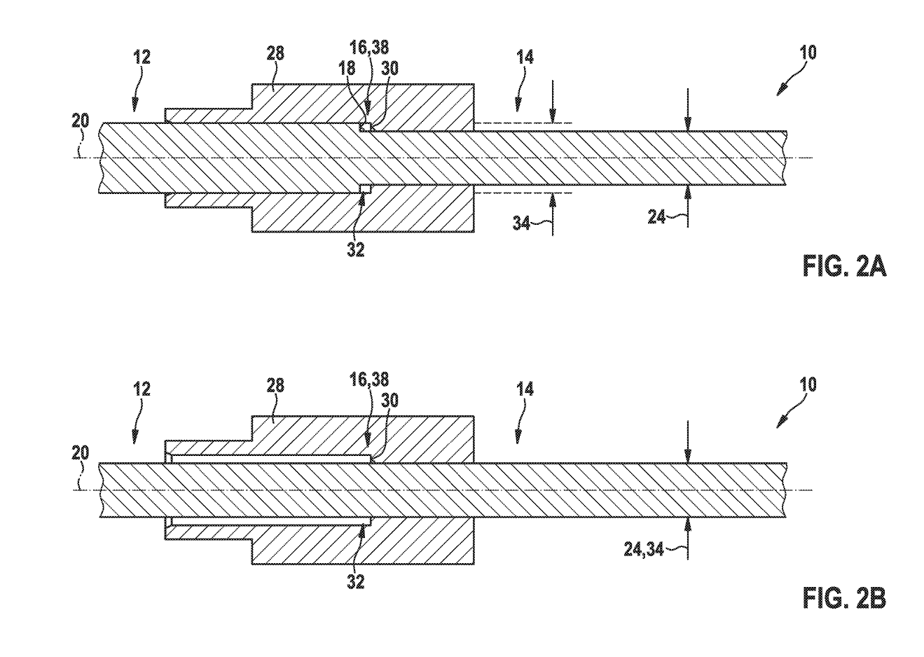

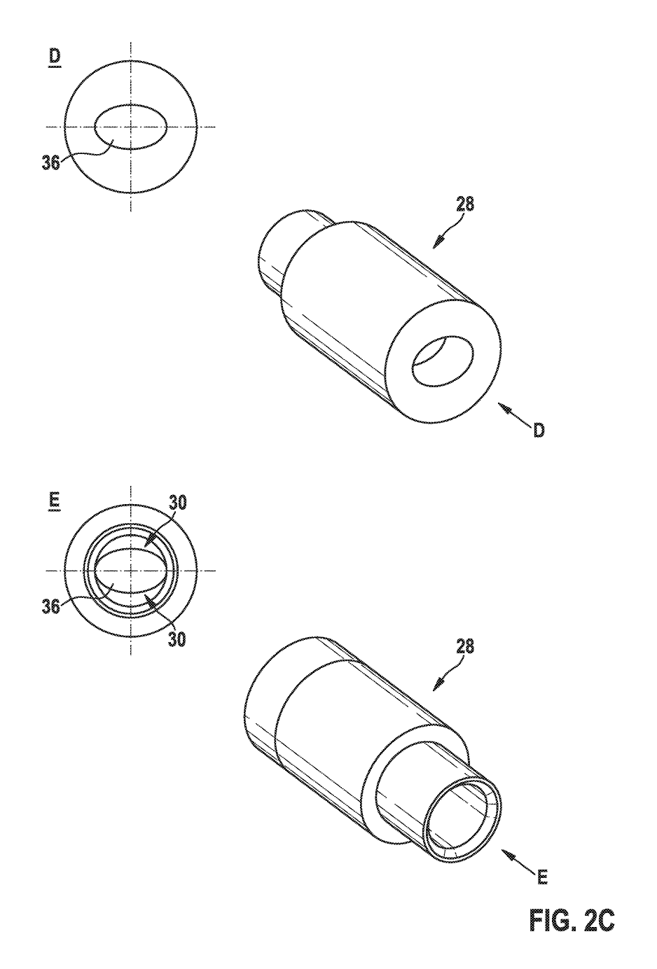

FIGS. 2A-2B show two sectional views offset 90° to one another through the same area of a second variant of the electrode implantation system, this area being essential to the present invention. One skilled in the art will appreciate that what was described for FIGS. 1A-1B also essentially applies for FIGS. 2A-2B, so that here only the differences will be discussed, and some of the reference numbers used are identical.

FIG. 2A shows a cross section of the inventive electrode implantation system in the area in which the electrode sleeve 28 is located on the elongated implantation tool 10. The implantable electrode 26 as a whole is no longer shown here. In this sample embodiment, the stop element 16 comprises a rotationally asymmetric area 32 around the common longitudinal axis 20. The cross section 34 of this rotationally asymmetric area 32 transverse to the common longitudinal axis 20 is larger than that of the distal section 14. Moving the electrode sleeve 28 along the common longitudinal axis 20 in the direction of the proximal section 12 can bring the complementary stop area 30 of the electrode sleeve 28 in contact with the stop surface 18 of the stop element 16 of the elongated implantation tool 10.

Compared with FIG. 2A, in FIG. 2B the elongated implantation tool 10 and the electrode sleeve 28 are rotated by 90° relative to one another around the common longitudinal axis 20. Therefore, it can be seen from FIG. 2B that the cross section 34 of the rotationally asymmetric area 32 of the stop element 16 is now no larger than the cross section 24 of the distal section 14. Thus, the rotationally asymmetric area 32 of the stop element 16 and the complementary stop area 30 form a type of lock-and-key system in which the stop surface 18 of the stop element 16 can be congruently superimposed on a corresponding cutout 36 (see FIG. 2C) in the complementary stop area 30. This orientation also allows the electrode sleeve 28 to be pushed beyond the stop element, completely onto the proximal section 12. Depending on the design of the rotationally asymmetric area 32 and the complementary stop area 30, there are a limited number of relative orientations of the implantation tool 10 and the electrode sleeve 28 around the common longitudinal axis 20 in which this is possible. In other words, there are then a limited number of relative orientations in which a projection of the complementary stop area 30 along the common longitudinal axis 20 and in the direction of the stop element 16 does not overlap in any place with the stop surface 18. It follows from FIG. 2A that the elongated implantation tool 10 in the proximal section 12 widens in the direction of the distal section 14 and tapers in the opposite direction.

FIG. 2C shows the electrode sleeve 28 isolated from the other elements of the inventive electrode implantation system. The views D and E of the electrode sleeve 28 (see FIG. 2C) show that the complementary stop area 30 comprises the cutout 36, which in this sample embodiment has an elliptical cross sectional shape. The stop element 16 from FIGS. 2A-2B (not shown in FIG. 2C), which represents a thickening 38 of the implantation tool 10, can then also have, in the area of the stop surface 18, an elliptical cross sectional shape transverse to the common longitudinal axis 20. Thus, the stop element 16 and the complementary stop area 30 fit together according to the lock-and-key principle.

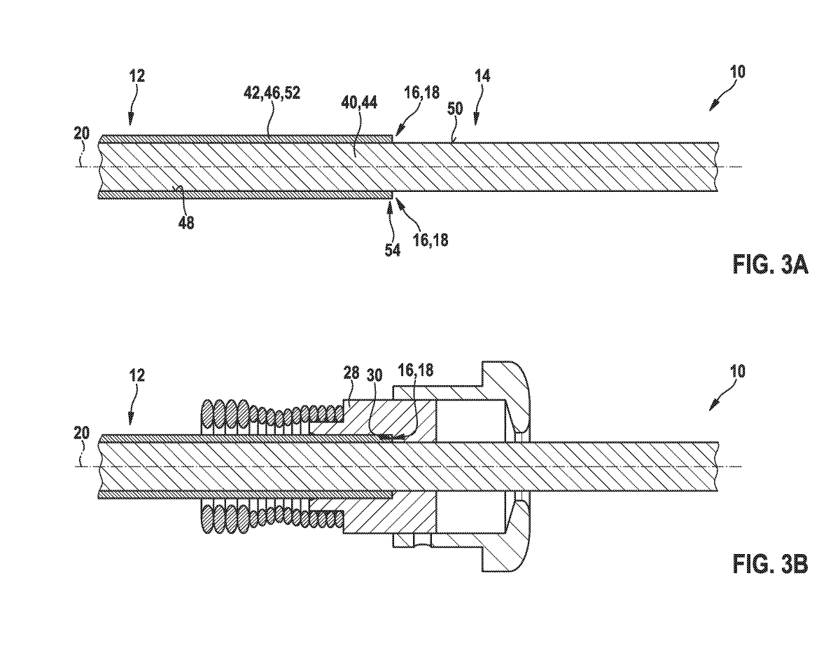

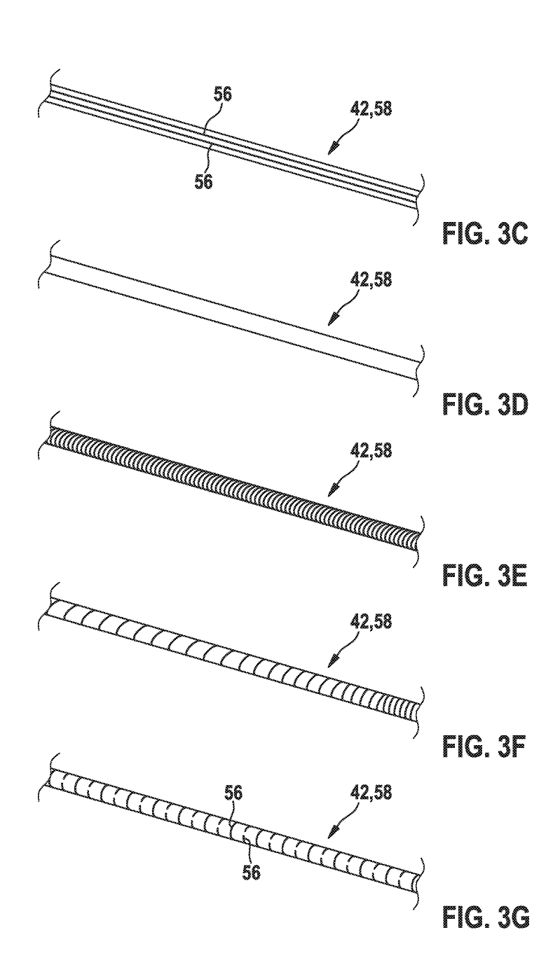

FIGS. 3A-3B show two sectional views through areas of a third variant of the electrode implantation system that are essential to the present invention. One skilled in the art will appreciate that if what was described for the preceding variants also applies for the embodiment according to FIGS. 3A and 3B, identical reference numbers are used.

FIGS. 3A-3B show an embodiment of the inventive electrode implantation system in which the elongated implantation tool 10 has two parts. FIG. 3A shows a cross-sectional view of the elongated implantation tool 10. This tool comprises an inner part 40 and an outer part 42. The inner part 40 is in the form of a flexible guide wire 44, and the outer part 42 is in the form of a mandrel 46. The outer part 42 comprises an inner lateral surface 48, and the inner part 40 comprises an outer lateral surface 50. The inner lateral surface 48 of the outer part 42 is pushed over, and can be moved on, the outer lateral surface 50 of the inner part 40. In this sample embodiment, the outer part 42 has a tubular geometry 52. Here, the outer part 42 also comprises the stop element 16. The stop surface 18 is provided on a distal end area 54 of the tubular geometry 52. Thus, the position of the stop element 16 on the inner part 40 is adjustable. Since the stop element 16 delimits the proximal section 12 and the distal section 14 from one another, the respective length of the proximal section 12 and the distal section 14 are flexibly adjustable. The distal section 14 is formed from the inner part 40 alone. By contrast, the proximal section 12 is formed by the inner part 40 and the outer part 42 together.

FIG. 3B shows a cross-sectional view of the elongated implantation tool 10 and the implantable electrode 26 with the electrode sleeve 28. It can be seen that in the state shown the stop surface 18 of the stop element 16 lies against the complementary stop area 30 of the electrode sleeve 28.

FIGS. 3C-3G show different embodiments of the outer part 42. FIG. 3C shows a sample embodiment of the outer part 42 that has a hollow cylindrical or also tubular geometry 52 and that additionally has elongated slots 56, which are made in the lateral surface of the outer part 42 and that extend along its longitudinal axis. According to FIG. 3G, each of the elongated slots 56 can also extend on a circumference or partial circumference around the lateral surface. FIG. 3D shows a sample embodiment of the outer part 42 that has purely tubular geometry 52. FIG. 3E shows a helical geometry 58 with a constant pitch. FIG. 3F shows a helical geometry 58 whose pitch decreases in the direction of the distal section 14 (not shown).

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An electrode implantation system comprising:
A) at least one elongated implantation tool that comprises a proximal section that is, at least in sections, in the form of a mandrel, and a distal section that is in the form of a flexible guide wire, the proximal and the distal sections being delimited from one another by a static or adjustable stop element comprising a stop surface facing the distal section; and
B) an implantable electrode with an electrode sleeve that has a stop area that is complementary to the stop surface and that faces the proximal section;
wherein the electrode sleeve and the elongated implantation tool are arranged so that they are, at least in sections, coaxial with one another, and can be moved relative to one another along and around a common longitudinal axis.

2. The electrode implantation system according to claim 1, wherein the stop element comprises a rotationally symmetric area around the common longitudinal axis, this rotationally symmetric area having a larger cross section transverse to the common longitudinal axis than the distal section does, and in each of the relative orientations of the elongated implantation tool and the electrode sleeve around the common longitudinal axis a projection of the complementary stop area along the common longitudinal axis overlaps with the stop surface.

3. The electrode implantation system according to claim 2, wherein the stop element is in the form of a spherical or ring-shaped thickening of the elongated implantation tool.

4. The electrode implantation system according to claim 1, wherein the stop element comprises a rotationally asymmetric area around the common longitudinal axis, this rotationally asymmetric area having a larger cross section transverse to the common longitudinal axis than the distal section does, and that there is a limited number of relative orientations of the elongated implantation tool and the electrode sleeve around the common longitudinal axis in which a projection of the complementary stop area along the common longitudinal axis does not overlap with the stop surface in any place.

5. The electrode implantation system according to claim 4, wherein the stop element is in the form of a thickening of the elongated implantation tool, the shape of this thickening's cross section transverse to the common longitudinal axis being polygonal or elliptical in the area of the stop surface.

6. The electrode implantation system according to claim 1, wherein the elongated implantation tool is made in two parts and comprises an inner part in the form of a flexible guide wire and an outer part in the form of a mandrel, and that the outer part is arranged so that an inner lateral surface of it can be moved on an outer lateral surface of the inner part, so that the distal section of the elongated implantation tool is formed by the inner part and the proximal section is formed by the inner part and the outer part, the outer part comprising the stop element.

7. The electrode implantation system according to claim 6, wherein the outer part has a tubular or helical geometry and the stop surface is provided on a distal end area of the tubular or helical geometry.

8. An elongated implantation tool, comprising:
a proximal section, at least sections of which are in the form of a mandrel;
a distal section that is in the form of a flexible guide wire; and
a static or adjustable stop element that delimits the proximal and the distal sections from one another and that comprises a stop surface facing the distal section.

9. The elongated implantation tool according to claim 8, wherein the stop element comprises a rotationally symmetric area around a longitudinal axis of the elongated implantation tool, this rotationally symmetric area having a larger cross section transverse to the longitudinal axis than the distal section does.

10. The elongated implantation tool according to claim 8, wherein the stop element comprises a rotationally asymmetric area around a longitudinal axis, this rotationally asymmetric area having a larger cross section transverse to the longitudinal axis than the distal section does.

11. The elongated implantation tool according to claim 10, wherein the stop element is in the form of a thickening of the elongated implantation tool, the shape of this thickening's cross section transverse to the longitudinal axis being polygonal or elliptical in the area of the stop surface.

12. The elongated implantation tool according to claim 8, wherein it is made in two parts and comprises an inner part in the form of a flexible guide wire and an outer part in the form of a mandrel, and that the outer part is arranged so that an inner lateral surface of it can be moved on an outer lateral surface of the inner part, so that the distal section is formed by the inner part and the proximal section is formed by the inner part and the outer part, the outer part comprising the stop element.

13. The elongated implantation tool according to claim 12, wherein the outer part has a tubular or helical geometry and the stop surface is provided on a distal end area of the tubular or helical geometry.

14. An implantable electrode comprising an electrode sleeve that has a stop area extending transverse to a longitudinal axis of the electrode sleeve.

15. An implantable electrode according to claim 14, wherein the stop area has a polygonal or elliptical cutout.
